(19) United States
(12) Patent Application Publication
(10) Pub. No.: US 2010/0166846 A1
(43) Pub. Date: Jul. 1, 2010

(54) MANAGEMENT OF MYOCLONUS WITH ORAL LIPOSOMAL REDUCED GLUTATHIONE

(76) Inventor: F. Timothy Guilford, Palo Alto, CA (US)

Correspondence Address: BROOKE SCHUMM III;Daneker, McIntire, Schumm, Prince, Goldstein et al, ONE NORTH CHARLES STREET, SUITE 2450, BALTIMORE, MD, 21201, US

(21) Appl. No.: 12/649,698

(22) Filed: Dec. 30, 2009

Related U.S. Application Data
(60) Provisional application No. 61/141,829, filed on Dec. 31, 2008.

(51) Int. Cl. A61K 9/127; A61K 38/06; A61P 25/00

(52) U.S. Cl. 424/450; 514/18

(57) ABSTRACT

The invention relates to a composition and method for the management of myoclonus and myoclonus and other illnesses related to mold toxin exposure.

The invention proposes the use of reduced glutathione encapsulated in a liposome (liposomal reduced glutathione) for the oral administration of a therapeutically effective amount to facilitate treatment of myoclonus. The oral liposomal reduced glutathione is also proposed for the removal of mold toxin and to improve symptoms in disease states related to exposure to mold toxins. The invention proposes a method of treatment combining an orally administrable preparation of liposomal glutathione with an anti-fungal agent.

### CONTINUATION DATA

This application claims the benefit of U.S. Provisional Application 61/141,829 filed Dec. 31, 2008 entitled “Management of myoclonus with oral liposomal reduced glutathione” and U.S. Provisional Application of the same name filed contemporaneously with this application having No. 61/290,903.

### FIELD OF INVENTION

This invention relates to a method of treatment of myoclonus and other illnesses related to mold toxin exposure after testing with a liposomal preparation of reduced glutathione and with a combination of a liposomal preparation of reduced glutathione and an anti-fungal agent.

### SUMMARY OF INVENTION

The invention relates to a composition and method for the management of myoclonus and myoclonus and other illnesses related to mold toxin exposure.

The invention proposes the use of reduced glutathione encapsulated in a liposome (liposomal reduced glutathione) for the oral administration of a therapeutically effective amount to facilitate treatment of myoclonus. The oral liposomal reduced glutathione is also proposed for the removal of mold toxin and to improve symptoms in disease states related to exposure to mold toxins. The invention proposes a method of treatment combining an orally administrable preparation of liposomal glutathione with an anti-fungal agent.

### TECHNICAL FIELD

The invention relates to the management of myoclonus and myoclonus related to mold toxin exposure with the delivery of reduced glutathione accomplished with the use of an oral liposomal preparation of reduced glutathione, uniquely designed to be absorbed a) across the mucosa of the nose, mouth, gastrointestinal tract, b) after topical application for transdermal, or c) by intravenous infusion of with or without liposome encapsulation.

### BACKGROUND OF INVENTION

The role of mold toxin in health has been difficult to assess as the relationship between mycotoxin and illness is often not appreciated unless the mold or yeast vector has been cultured from the blood of an infected individual. Individuals with the dissemination in the blood of a yeast or mold are often immune compromised. Diagnosis of such an infection requires therapy with systemic antifungal agents. Toxins from molds or yeast are generally referred to as mycotoxins.

Illness related to the accumulation of toxins from mold or yeast is generally reported after the finding of toxin contamination in food for humans and more commonly in animal feed (1).

Indoor exposure to toxigenic mold has been proposed, but the ability to establish the presence of mold using reproducible clinical laboratory techniques has been lacking, so the concept remains largely conjectural and very controversial. Exposure to toxigenic mold has been conjectured to cause asthma, airway irritation and bleeding, dizziness, and impaired memory and concentration problems (2).

Only recently, a commercial test for presence of mold toxin in body fluid has become available from Real Time Laboratories, LLC, 13016 Bee Street, Suite 203, Dallas, Tex. 75234. Mold toxins are concentrated and excreted in urine, so urine is the preferred fluid for testing for the presence of mold toxin from an individual, assuming a test could be available. With the novel ability to assess and assay mold toxin in a commercially reasonably manner, the need for and measurability of a therapy for these toxins becomes feasible and necessary. While systemic antifungal therapies are available, the possibility of speeding the removal of toxins would be a significant adjunct to the management of mycotoxin related illness.

The inventor has conceived that surprising benefit of oral liposomal glutathione might be useful to be administered to an animal incapacitated by myoclonus and to the moderately affected human companion of the animal. Upon administration, the inventor has discovered significant lessening of the individual's symptoms as reviewed in Case

### EXAMPLES 1 and 2

Myoclonus is a hyperkinetic movement disorder characterized by quick, involuntary jerks or movements. These movements can be localized to certain muscle groups or may be generalized to an area such as extremity. Myoclonus encompasses a vast range of etiologies and widespread anatomic locations. Treatment of myoclonus has been complicated by the fact that there is no known specific etiology. The majority of the literature is comprised of case reports (3). The inventor theorizes the myoclonus symptoms were related to mold toxins.

Glutathione is known for its role in biochemical detoxification (4). However, glutathione has not been commercially available in a form usable for ordinary patient treatment outside of a clinical setting despite having been reported from cell culture studies as far back as 1979 to neutralize the aflatoxin B1 (5). However, there has not been a report or suggestion of the use of glutathione for the management of myoclonus or mycotoxin related myoclonus.

Myoclonus currently has no specific medication for treatment and, given the uncertain pathology and etiology, a treatment modality or paradigm, has not been developed. Mycotoxin related myoclonus has not been previously reported. The use of oral liposomal reduced glutathione is referenced for the treatment of myoclonus and specifically mycotoxin related myoclonus.

### OBJECTS OF THE INVENTION

A primary object of the invention is a composition and method of treating myoclonus by a liposomal reduced glutathione capable of effective oral administration while achieving the desired intracellular results specifically meaning capable of being absorbed a) across the mucosa of the nose, mouth, gastrointestinal tract, b) after topical application for transdermal, or c) by intravenous infusion of with or without liposome encapsulation. A corollary and critical characteristic to liposomal reduced glutathione being administrable orally is that the method use a composition and the composition be one which is capable of long term storage without refrigeration.

### PREFERRED MODES OF INVENTION

Liposomal reduced glutathione may also be considered in combination with antifungal medications for the management of fungal related illnesses and exposures, particularly if mycotoxins are found to be present in one or more body fluids or tissues of an individual.

Reference is made to earlier applications of Guilford: U.S. Application published as US 20040022873 filed on Nov. 9, 2002 entitled Systemic Administration Of Glutathione Or Precursor Such As NAC As An Adjunct In The Treatment Of Bacillus Anthracis Exposure Or Infection, of provisional application 60/371,590 filed on Apr. 11, 2002 entitled Use Of Glutathione Precursor In The Treatment Of Smallpox And The Use Of Glutathione Precursor In The Treatment Of Radiation Exposure, The Use Of The Combination Of Glutathione Precursor And DHEA For The Treatment Of Smallpox And Other Viruses, all of which are incorporated by reference herein, and Liposomal Formulation for Oral Administration of Glutathione (Reduced) U.S. Utility appl. Ser. No. 11/163,979 filed Nov. 6, 2005, and provisional applications as to which the latter application claims benefit and/or continuation in part status.

Antifungal medications include:

Polyene antifungals. These are not absorbed orally:

### Amphotericin, Nystatin, Griseofulvin, Flucytosine, Terbinafine, Caspofungin.

Statins have also been shown to have antifungal qualities, possibly due to the ability of statins to inhibit production of isoprenylated proteins that are essential to fungi (6). Statins include: Simvastatin, fluvastatin, lovastatin, atorvastatin, rosuvastatin, pravastatin.

Imidazoles are taken orally:

Miconazole—(Miconazole nitrate), Ketoconazole, Clotrimazole—marketed as Lotrimin or Lotrimin AF (and Canesten in the UK). Econazole, Bifonazole, Butoconazole, Fenticonazole, Isoconazole, Oxiconazole, Sertaconazole—marketed as Ertaczo in North America. Sulconazole, Tioconazole

Triazoles are taken orally:

### Fluconazole, Itraconazole, Isavuconazole, Ketoconazole, Ravuconazole, Posaconazole, Voriconazole, Terconazole.

Allylamines

Allylamines inhibit the enzyme squalene epoxidase, another enzyme required for ergosterol synthesis:

Terbinafine—marketed as “Lamisil” in North America, Australia, the UK, Germany and the Netherlands. Amorolfine, Naftifine—marketed as “Naftin” in North America. Butenafine—marketed as Lotrimin Ultra.

Echinocandins

Echinocandins inhibit the synthesis of glucan in the cell wall, probably via the enzyme 1,3-β glucan synthase:

### Anidulafungin, Caspofungin, Micafungin

Other

Ciclopirox—(ciclopirox olamine), Tolnaftate—fungicidal, marketed as Tinactin, Desenex, Aftate, as well as other names. Undecylenic acid—organic unsaturated fatty acid derived from natural castor oil, fungistatic as well as anti-bacterial and anti-viral.

Flucytosine, or 5-fluorocytosine, is an antimetabolite.
Griseofulvin—binds to polymerized microtubules and inhibits fungal mitosis.
Haloprogin—discontinued due to the emergence of more modern antifungals with fewer side effects

The preferred combination is 200 mg of itraconazole orally per day for 2 to 16 weeks in combination with oral liposomal reduced glutathione 422 mg (1 teaspoon) twice a day.

The preferred combination is 200 mg of voriconazole orally per day for 2 to 16 weeks in combination with oral liposomal reduced glutathione 422 mg (1 teaspoon) twice a day.

Intranasal antifungal therapy in the form of irrigation or topical intranasal spray may also be used in combination with oral liposomal reduced glutathione. The objective of this therapy is to reduce the presence and growth of fungal material in the nose and adjacent sinuses.

The dose of oral liposomal reduced glutathione is oral liposomal reduced glutathione 422 mg (1 teaspoon) twice a day.

The preferred therapeutic for intranasal therapy is
0.3% (3 mg/mL) amphotericin B suspension in a nasal spray twice a day (total volume 800 μl) in each nostril, twice a day, during 4-16 weeks.
Another preferred method for the nasal spray is to use 100 mg of fluconazole in 500 ml of normal saline solution administered as 5 sprays (0.5 cc/spray) in each nostril twice daily.
Another preferred method for the nasal spray is to use Itraconazole 0.1% Nasal Spray 5 sprays each nostril twice a day.

Plain glutathione used orally is not an option for this therapy as plain glutathione is not absorbed after oral ingestion in humans (7). A rat study of the removal of a radio-tagged metal (CO-60) from the liver, performed at Pacific Northwest National Laboratory with oral liposomal reduced glutathione confirms this observation. The tissue from the control animals (water) served as the 100% of the toxin remaining in the tissue. The animals receiving:
a. Control (water only) showed 90% of the toxin remained=0% removal
b. Plain glutathione, oral, in water showed 90% of the toxin remained=10% removal.
c. Intravenous glutathione showed 30% of the toxin remaining=70% removal.
d. Liposomal reduced glutathione showed 40% of the toxin remaining=60% removal.

The data from this study is consistent with the observation that liposomal glutathione is almost as effective as intravenous glutathione in mobilizing the toxin. The plain glutathione has little if any absorption or efficacy.

Oral liposomal reduced glutathione that is uniquely designed to be absorbed a) across the mucosa of the nose, mouth, gastrointestinal tract, b) after topical application for transdermal, or c) by intravenous infusion of with or without liposome encapsulation is prepared under the method and according to the composition described as follows:

### DETAILED DESCRIPTION OF THE PREFERRED EMBODIMENTS

### EXAMPLE 1

Oral liposomal reduced glutathione Drink or Spray 2500 mg Per Ounce

TABLE 1

| | % w/w |
|---|---|
| Deionized Water | 74.4 |
| Glycerin | 15.00 |
| Lecithin | 1.5 |
| Citrus Seed Extract | 0.50 |
| Potassium sorbate | 0.10 |
| Glutathione | 8.5 |
| (reduced) | |

A lipid mixture having components lecithin and glycerin were commingled in a large volume flask and set aside for compounding

In a separate beaker, water, glycerin, and reduced glutathione were mixed and heated to 50 degrees C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally a spoilage retardant such as potassium sorbate or BHT would be added. The solution would be placed in appropriate dispenser for ingestion as a liquid or administration as a spray.

Analysis of the preparation under an optical light microscope with polarized light at 400.times. magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. No. 5,891,465 are incorporated into this description. The inventor, for all the examples, prefers to purchase the liposomal embodiment and application with reduced glutathione from Biozone, Inc., 580 Garcia Ave, Pittsburg, Calif. (USA) 94565.

### EXAMPLE 1A

Liposomal reduced glutathione Drink or Spray 2500 mg Per Ounce or Form Suitable for Encapsulation or Gel

TABLE 2

| | % w/w |
|---|---|
| Deionized Water | 74.4 |
| Glycerin | 15.00 |
| Lecithin | 1.5 |
| Citrus Seed Extract | 0.50 |
| Potassium sorbate | 0.10 |
| Glutathione | 8.5 |
| (reduced) | |

A lipid mixture having components lecithin, ethyl alcohol and glycerin were commingled in a large volume flask and set aside for compounding. The lecithin is normally derived from soybeans.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, citrus seed extract would be added. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400.times. magnification confirmed presence of both multilamellar lipid vesicles (MLV) and unilamellar lipid vesicles.

The preferred embodiment includes the variations of the amount of reduced glutathione to create less concentrated amounts of glutathione. The methods of manufacture described in Keller et al U.S. Pat. No. 5,891,465 are incorporated into this description.

### EXAMPLE 2

Liposomal Reduced Glutathione

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.

The liposome-capsule unit containing biologically encapsulated material can be taken in addition to orally, used for topical unit-of-use application, or other routes of application such as intra-ocular, intranasal, rectal, or vaginal.

The composition of examples 1 and 2 may be utilized in the encapsulated embodiment of this invention.

Gelatin capsules have a lower tolerance to water on their interior and exterior. The usual water tolerance for a soft gel capsule is 10% on the interior. The concentration of water in a liposome formulation can range from 60-90% water. An essential component of the present invention is the formulation of a liposome with a relatively small amount of water, in the range of 5-10%. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% range.

The methods described by Keller et al, U.S. Pat. No. 6,726,924 are incorporated in this description.

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting capsule contains 10 mg CoQ10. Filling of gel caps on a large scale is best with the rotary die method or others such as the Norton capsule machine.

### EXAMPLE 3

Glutathione LipoCap Formulation

TABLE 3

| Ingredient | Concentration (%) |
|---|---|
| Sorbitan Oleate | 2.0 |
| Reduced Glutathione | 89.8 |
| Purified Water | 4.0 |
| Potassium Sorbate | 0.2 |
| Polysorbate 20 | 2.0 |
| Phospholipon90 (DPPC) | 2.0 |

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting one gram capsule contains 898 IU of Vitamin E500 mg. Large scale manufacturing methods for filling gel caps, such as the rotary die process, are the preferred method for commercial applications.

Embodiment number three of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description. The result is a product having lecithin derived from soybeans that is hydroxylated. The product is capable of very long term (14 months and greater) storage without refrigeration while remaining stable which is another unique characteristic of the invention.

Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of P. sub.a (P.sub.a references the surface packing parameter) between about 0.84 and 0.88, a P.sub.v (P.sub.v references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution.

### CASE EXAMPLE 1

A 2 year old, 60 pound male canine presented with idiopathic myoclonus in the thoracic region that was severe enough to cause disruption of mobility and severe, persisting incapacitation to the extent that euthanizing the animal was considered. Evaluation by a neurologic veterinarian revealed no specific etiology. The symptoms were managed initially with valium and then methocarbolmol, neither of which was tolerated by the animal. Subsequent testing through a commercial lab using mycotoxin toxin-specific monoclonal antibody tests revealed a positive urine sample for aflatoxin, tricothecene and ochratoxin. The animal's indoor environment was found to culture Stachybotris chatarum, which is known to produce tricothecene toxins and Aspergillus, which is known to produce aflatoxin. The animal was placed on a dietary supplement of oral liposomal reduced glutathione 1 teaspoon, which contains 400 mg glutathione twice a day. Within 7 days the myoclonus symptoms resolved entirely and the animal is stable and comfortable off of the medications.

### CASE EXAMPLE 2

A 28 year old woman in previously good health developed mild myoclonus of arms with movement and some difficulty with memory. The individual's indoor environment was found to culture Stachybotris chatarum, which is known to produce tricothecene toxins and Aspergillus, which is known to produce aflatoxin. Subsequent testing through a commercial lab using mycotoxin toxin-specific monoclonal antibody tests revealed a positive urine sample for aflatoxin and tricothecene. A visual contrast test (8) used to identify mild aberration of visual performance by the ability to distinguish the direction of tiny light and grey lines was used as an assessment. The individual reports being able to identify the correct answer only 7% of the time. After using oral liposomal reduced glutathione for 10 days the individual reports that the myoclonus and the memory difficulty was resolved and that her visual acuity test now able to identify the correct answer 93% of the time.

The invention is applicable to neurodegenerative diseases that are related to mycotoxin-related nerve damage, which include Amyotrophic Lateral Sclerosis (ALS). The preferred embodiment would be to administer the combination of 200 mg of voriconazole orally per day for 2 to16 weeks in combination with oral liposomal reduced glutathione 422 mg (1 teaspoon) twice a day. An alternative embodiment for neurodegenerative diseases that are related to mycotoxin-related nerve damage is to administer 200 mg of itraconazole orally per day for 2 to16 weeks in combination with oral liposomal reduced glutathione 422 mg (1 teaspoon) twice a day.

The preferred combination is 200 mg of voriconazole orally per day for 2 to16 weeks in combination with oral liposomal reduced glutathione 422 mg (1 teaspoon) twice a day.

The invention is not meant to be limited to the disclosures, including best mode of invention herein, and contemplates all equivalents to the invention and similar embodiments to the invention for humans and mammals and veterinary science. Applications by the inventor Guilford referenced herein are adopted by reference to the extent necessary needed to supplement the specification and to understand the invention and claims.

1. Sorenson W G, Gerberick G F, Lewis D M, Castranova V. Toxicity of mycotoxins for the rat pulmonary macrophage in vitro. Environmental health perspectives. 1986;66:45-53. Cited in PubMed; 2423320.

2. Kilburn K H. Indoor mold exposure associated with neurobehavioral and pulmonary impairment: a preliminary report. Archives of environmental health. 2003;58(7):390-8. Cited in PubMed; 15143851.

3. Chang V C, Frucht S J. Myoclonus. Current treatment options in neurology. 2008;10(3):222-9. Cited in PubMed; 18579026.

4. Ketterer B, Coles B, Meyer D J. The role of glutathione in detoxication. Environmental health perspectives. 1983;49:59-69. Cited in PubMed; 6339228.

5. Emerole G O, Neskovic N, Dixon R L. The detoxication of aflatoxin B1 with glutathione in the rat. Xenobiotica; the fate of foreign compounds in biological systems. 1979;9(12):737-43. Cited in PubMed; 575250.

6. Roehr B. Statins Show Antimicrobial Activity in Vitro. Medscape Today [serial on the Internet]. 2007; (Sept. 30): Available from:

http://www.medscape.com/viewarticle/563088.

7. Witschi A, Reddy S, Stofer B, Lauterburg B H. The systemic availability of oral glutathione. European journal of clinical pharmacology. 1992;43(6):667-9. Cited in PubMed; 1362956.

8. Rosen G, Schaller J. Your Guide to Mold Toxins. Moczik B, editor. Tampa, Fla.: Hope Academic Press; 2006.

I claim:

1. A method of treatment of patients having myoclonus-related symptoms as a result of mold toxin exposure comprising:
administration of an oral liposomal preparation of reduced glutathione.

2. A method of treatment of patients having myoclonus-related symptoms as a result of mold toxin exposure comprising:
administration of an oral liposomal preparation of reduced glutathione formulated from said reduced glutathione in liposomal formulation being formulated in a process whose temperature does not exceed 50 degrees C., and which process utilizes mixing of a first container of lecithin and glycerin, and a second container of components of at least deionized water and glutathione (reduced) and upon mixing at a temperature not in excess of 50 degrees, said reduced glutathione in liposomal formulation thereby being capable of storage at room temperature for at least one month with at least 50% of original reduced glutathione.

3. A method of treatment of patients having myoclonus-related symptoms as a result of mold toxin exposure comprising:
administration of an oral liposomal preparation of reduced glutathione in a form which can be taken orally or absorbed across the mucosa of the nose, mouth, gastrointestinal tract.

4. A method of treatment of patients having myoclonus-related symptoms as a result of mold toxin exposure comprising:
administration of an oral liposomal preparation of reduced glutathione in a form which can be absorbed by topical application for transdermal administration.

5. A method of treatment of patients having myoclonus-related symptoms as a result of mold toxin exposure comprising:
administration of reduced glutathione by intravenous infusion

6. The method according to claims 1-5 further comprising:
administration of a pharmaceutical agent having anti-fungal properties.

7. The method according to claims 1 through 3, further comprising:
intranasal administration of a pharmaceutical agent having anti-fungal properties.

8. A method of treatment of patients having myoclonus-related symptoms as a result of mold toxin exposure comprising:
administration of an oral liposomal preparation of reduced glutathione; and
administration of itraconazole.

9. A method of treatment of patients having myoclonus-related symptoms as a result of mold toxin exposure comprising:
administration of an oral liposomal preparation of reduced glutathione; and
administration of amphoteracin.

10. A method of treatment of patients having myoclonus-related symptoms as a result of mold toxin exposure comprising:
administration of an oral liposomal preparation of reduced glutathione; and
administration of fluconazole.

11. A method of treatment of patients having myoclonus-related symptoms as a result of mold toxin exposure comprising:
administration of an oral liposomal preparation of reduced glutathione; and
administration of voriconazole.

12. A method of treatment for patients having myoclonus-related neurodegenerative disease as a result of mold toxin exposure comprising:
administration of an oral liposomal preparation of reduced glutathione; and
administration of voriconazole.

13. The method according to claims 1, and 3 through 5, and 8 though 12, further comprising:
said oral liposomal preparation of reduced glutathione being formulated in a process whose temperature does not exceed 50 degrees C., and which process utilizes mixing of a first container of lecithin and glycerin, and a second container of components of at least deionized water and glutathione (reduced) and upon mixing at a temperature not in excess of 50 degrees, said reduced glutathione in liposomal formulation thereby being capable of storage at room temperature for at least one month with at least 50% of original reduced glutathione.
